SEQUENCE LISTING (23 sequences)

SEQ ID NO: 1 - protein, 320 aa, Homo sapiens
  MISC_FEATURE: Mature Human CNTFR alpha
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGS

SEQ ID NO: 2 - protein, 372 aa, Homo sapiens
  MISC_FEATURE: Human CNTFR alpha
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 3 - protein, 320 aa, Mus musculus
  MISC_FEATURE: Mature Mouse CNTFR alpha
QKHSPQEAPHVQYERLGADVTLPCGTASWDAAVTWRVNGTDLAPDLLNGSQLILRSLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNTTAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAPETTTSTTSSLAPPPTTKICDPGELGS

SEQ ID NO: 4 - protein, 372 aa, Mus musculus
  MISC_FEATURE: Mouse CNTFR alpha
MAASVPWACCAVLAAAAAAVYTQKHSPQEAPHVQYERLGADVTLPCGTASWDAAVTWRVNGTDLAPDLLNGSQLILRSLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNTTAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAPETTTSTTSSLAPPPTTKICDPGELGSGGGPSILFLTSVPVTLVLAAAAATANNLLI

SEQ ID NO: 5 - protein, 198 aa, Homo sapiens
  MISC_FEATURE: Mature Human CLC
LNRTGDPGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPPAAAVTLHLGAHGF

SEQ ID NO: 6 - protein, 225 aa, Homo sapiens
  MISC_FEATURE: Human CLC
MDLRAGDSWGMLACLCTVLWHLPAVPALNRTGDPGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPPAAAVTLHLGAHGF

SEQ ID NO: 7 - protein, 198 aa, Mus musculus
  MISC_FEATURE: Mature Mouse CLC
LNRTGDPGPGPSIQKTYDLTRYLEHQLRSLAGTYLNYLGPPFNEPDFNPPRLGAETLPRATVNLEVWRSLNDRLRLTQNYEAYSHLLCYLRGLNRQAATAELRRSLAHFCTSLQGLLGSIAGVMATLGYPLPQPLPGTEPAWAPGPAHSDFLQKMDDFWLLKELQTWLWRSAKDFNRLKKKMQPPAASVTLHLEAHGF

SEQ ID NO: 8 - protein, 225 aa, Mus musculus
  MISC_FEATURE: Mouse CLC
MDLRAGDSWGMLACLCTVLWHLPAVPALNRTGDPGPGPSIQKTYDLTRYLEHQLRSLAGTYLNYLGPPFNEPDFNPPRLGAETLPRATVNLEVWRSLNDRLRLTQNYEAYSHLLCYLRGLNRQAATAELRRSLAHFCTSLQGLLGSIAGVMATLGYPLPQPLPGTEPAWAPGPAHSDFLQKMDDFWLLKELQTWLWRSAKDFNRLKKKMQPPAASVTLHLEAHGF

SEQ ID NO: 9 - protein, 385 aa, Homo sapiens
  MISC_FEATURE: Mature Human CLF
AHTAVISPQDPTLLIGSSLLATCSVHGDPPGATAEGLYWTLNGRRLPPELSRVLNASTLALALANLNGSRQRSGDNLVCHARDGSILAGSCLYVGLPPEKPVNISCWSKNMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGACEPRGGEPSSGPVRRELKQFLGWLKKHAYCSNLSFRLYDQWRAWMQKSHKTRNQDEGILPSGRRGTARGPAR

SEQ ID NO: 10 - protein, 422 aa, Homo sapiens
  MISC_FEATURE: Human CLF
MPAGRRGPAAQSARRPPPLLPLLLLLCVLGAPRAGSGAHTAVISPQDPTLLIGSSLLATCSVHGDPPGATAEGLYWTLNGRRLPPELSRVLNASTLALALANLNGSRQRSGDNLVCHARDGSILAGSCLYVGLPPEKPVNISCWSKNMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGACEPRGGEPSSGPVRRELKQFLGWLKKHAYCSNLSFRLYDQWRAWMQKSHKTRNQDEGILPSGRRGTARGPAR

SEQ ID NO: 11 - protein, 392 aa, Mus musculus
  MISC_FEATURE: Mature Mouse CLF
VPRGGSGAHTAVISPQDPTLLIGSSLQATCSIHGDTPGATAEGLYWTLNGRRLPSELSRLLNTSTLALALANLNGSRQQSGDNLVCHARDGSILAGSCLYVGLPPEKPFNISCWSRNMKDLTCRWTPGAHGETFLHTNYSLKYKLRWYGQDNTCEEYHTVGPHSCHIPKDLALFTPYEIWVEATNRLGSARSDVLTLDVLDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGVCEPRGGEPSSGPVRRELKQFLGWLKKHAYCSNLSFRLYDQWRAWMQKSHKTRNQDEGILPSGRRGAARGPAG

SEQ ID NO: 12 - protein, 425 aa, Mus musculus
  MISC_FEATURE: Mouse CLF
MPAGRPGPVAQSARRPPRPLSSLWSPLLLCVLGVPRGGSGAHTAVISPQDPTLLIGSSLQATCSIHGDTPGATAEGLYWTLNGRRLPSELSRLLNTSTLALALANLNGSRQQSGDNLVCHARDGSILAGSCLYVGLPPEKPFNISCWSRNMKDLTCRWTPGAHGETFLHTNYSLKYKLRWYGQDNTCEEYHTVGPHSCHIPKDLALFTPYEIWVEATNRLGSARSDVLTLDVLDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGVCEPRGGEPSSGPVRRELKQFLGWLKKHAYCSNLSFRLYDQWRAWMQKSHKTRNQDEGILPSGRRGAARGPAG

SEQ ID NO: 13 - protein, 119 aa, Homo sapiens
  MISC_FEATURE: Mature Human BDNF
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 14 - protein, 137 aa, Homo sapiens
  MISC_FEATURE: Human BDNF
MRILLLTMVISYFGCMKAHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 15 - protein, 119 aa, Mus musculus; identical to SEQ ID NO: 13
  MISC_FEATURE: Mature Mouse BDNF

SEQ ID NO: 16 - protein, 137 aa, Mus musculus; identical to SEQ ID NO: 14
  MISC_FEATURE: Mouse BDNF

SEQ ID NO: 17 - protein, 119 aa, Homo sapiens [sequence omitted]

SEQ ID NO: 18 - protein, 257 aa, Homo sapiens
  MISC_FEATURE: Human NT-3
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 19 - protein, 119 aa, Mus musculus [sequence omitted]

SEQ ID NO: 20 - protein, 258 aa, Mus musculus
  MISC_FEATURE: Mouse NT-3
MSILFYVIFLAYLRGIQGNSMDQRSLPEDSLNSLIIKLIQADILKNKLSKQMVDVKENYQSTLPKAEAPREPEQGEATRSEFQPMIATDTELLRQQRRYNSPRVLLSDSTPLEPPPLYLMEDYVGNPVVANRTSPRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 21 - protein, 526 aa, Artificial Sequence
  Mature human CNTFR alpha/CLC fusion protein
  MISC_FEATURE (1)..(323): CNTFR alpha
  MISC_FEATURE (324)..(337): Linker
  MISC_FEATURE (338)..(526): CLC
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDLEGGGGSGGGGSLELNRTGDPGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPPAAAVTLHLGAHGF

SEQ ID NO: 22 - protein, 548 aa, Artificial Sequence
  Human CNTFR alpha/CLC fusion protein
  MISC_FEATURE (1)..(22): Leader sequence
  MISC_FEATURE (23)..(336): CNTFR alpha
  MISC_FEATURE (337)..(350): Linker
  MISC_FEATURE (351)..(548): CLC
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDLEGGGGSGGGGSLELNRTGDPGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPPAAAVTLHLGAHGF

SEQ ID NO: 23 - protein, 14 aa, Artificial Sequence
  Linker Sequence
LEGGGGSGGGGSLE

(19) United States
(12) Patent Application Publication
(10) Pub. No.: US 2022/0324921 A1
(43) Pub. Date: Oct. 13, 2022

(54) METHODS AND COMPOSITIONS FOR THE TREATMENT OF ALS

(71) Applicant: University of Cincinnati, Cincinnati, OH (US)

(72) Inventor: Alexander John MacLennan, Cincinnati, OH (US)

(73) Assignee: University of Cincinnati, Cincinnati, OH (US)

(21) Appl. No.: 17/639,949

(22) PCT Filed: Sep. 3, 2020
(86) PCT No.: PCT/US2020/049123

§ 371 Date: Mar. 3, 2022

Related U.S. Application Data
(60) Provisional application No. 62/895,052, filed on Sep. 3, 2019.

(51) Int. Cl. C07K 14/435; C12N 15/861; A61P 25/00

(52) U.S. Cl. CPC C07K 14/435; C12N 15/861; A61P 25/00

(57) ABSTRACT

Compositions including modified adeno-associated virus (AAV) vectors comprising a recombinant AAV (rAAV)-based genome are provided herein, wherein the rAAV-based genome includes one or more of: a brain derived neurotrophic factor (BDNF)-encoding cDNA insert; or a neurotrophin-3 (NT-3)-encoding cDNA insert. Also provided are methods of treating motor neuron degenerative disorders, such as amyotrophic lateral sclerosis (ALS), by administering the disclosed compositions.

### CROSS-REFERENCE TO RELATED APPLICATIONS

This application claims priority to U.S. Provisional Application Ser. No. 62/895,052, filed Sep. 3, 2019, the entire contents of which are incorporated herein by reference.

### TECHNICAL FIELD

The present disclosure relates to compositions and methods for the treatment of motor neuron degenerative disorders and, more particularly, for the treatment of amyotrophic lateral sclerosis (ALS).

### BACKGROUND

Amyotrophic lateral sclerosis (ALS) is an adult-onset, neurodegenerative, invariably terminal disease with death typically occurring within 2-3 years of symptom onset and an estimated annual cost of over a billion dollars in the United States alone. There is no known cure for ALS and only two FDA approved treatments: riluzole, approved in 1995, and edaravone, approved in 2017. However, neither riluzole nor edaravone has been shown to prolong patient survival longer than three months.

ALS patients do not develop overt symptoms until late in the underlying disease process. Even with tertiary care centers and recent advances, diagnosis still takes approximately one year following symptom onset, further delaying treatment for all but the rare genetically diagnosed cases. Hence, any broadly useful ALS treatment must be effective when initiated very late in the underlying disease after the ALS patient has been diagnosed. While the mechanism(s) underlying ALS are still being determined, they undoubtedly involve a series of molecular and cellular events. Interventions effective against early steps in this process would not necessarily be expected to be effective against the later steps occurring when patients are finally diagnosed. Therefore, it is not surprising that interventions developed by treating ALS mice at earlier disease stages have a history of failure in clinical trials.

Work with human ALS cell culture and model mice containing mutant copper zinc superoxide dismutase 1 (SOD1) indicates that multiple cell types, including glia, are involved in the motor neuron (MN) degeneration responsible for ALS symptoms and death. Many cellular and molecular causes have been proposed, but if a common cause is responsible for all ALS, it has yet to be established.

Indirect data have suggested that ciliary neurotrophic factor (CNTF) receptors broadly protect MNs from a wide variety of insults, including ALS. But CNTF's poor tissue penetration and short half-life inhibit neuromuscular access following systemic injection.

Neuromuscular CNTF receptors are restricted to MNs and muscle, with muscle being a particularly promising target since expression in muscle, unlike MNs, can be specifically modified in humans with approved gene therapy techniques. Moreover, the present investigator's muscle-specific knockdown studies surprisingly found endogenous muscle CNTF receptor α (CNTFRα), the essential ligand binding subunit of the CNTF receptor, inhibits ALS in a wide variety of genetically-defined ALS models. This suggested that the increased muscle CNTFRα found in all ALS models (and reported for human ALS) is a broadly effective anti-ALS response to the disease that could be enhanced to further inhibit disease progression.

Previous studies increased muscle CNTFRα expression in SOD1^{G93A} ALS mice with an adeno-associated virus (AAV) vector. The data showed that intramuscular injection of an adeno-associated virus (AAV) gene delivery vector comprising a recombinant AAV-based genome comprising a cDNA insert encoding ciliary neurotrophic factor receptor alpha (CNTFRα), cardiotrophin-like cytokine factor 1 (CLC), and/or cytokine receptor-like factor 1 (CLF) is effective to increase endogenous neuroprotective mechanisms and inhibit progression of ALS. See WO 2017/0173234, incorporated herein by reference in its entirety. This treatment extended survival and delayed motor decline in the mouse model, without side effect.

A need exists for further compositions and methods for the treatment of ALS, particularly broadly effective therapeutic options that prolong survival and abate disease progression independent of the specific cause of ALS.

### SUMMARY

Accordingly, provided herein are methods and compositions for the treatment of motor neuron degenerative disorders, including ALS. Without being bound by theory, it is believed that the disclosed methods and compositions function in the ALS patient by enhancing endogenous mechanisms for the protection of motor neurons.

In one embodiment, a method of treating a subject suffering from a motor neuron degenerative disorder is provided, the method comprising administering to the subject one or more modified adeno-associated virus (AAV) vectors comprising a recombinant AAV (rAAV)-based genome, wherein the rAAV-based genome comprises one or more of: a cDNA insert encoding brain derived neurotrophic factor (BDNF); or a cDNA insert encoding neurotrophin-3 (NT-3).

In another embodiment, a pharmaceutical composition is provided, comprising: one or more muscle-tropic modified AAV vectors, each of said AAV vectors comprising an rAAV genome, each of said rAAV genomes engineered to comprise: (i) one or more of: a cDNA insert encoding BDNF or a cDNA insert encoding NT-3; and (ii) a promoter; and at least one pharmaceutically-acceptable excipient.

In another embodiment, a modified adeno-associated virus (AAV) vector comprising a plurality of recombinant AAV (rAAV)-based genomes is provided, wherein the rAAV genomes comprise at least one of: a cDNA insert encoding BDNF or a cDNA insert encoding NT-3; and one or more of a cDNA insert encoding ciliary neurotrophic factor receptor alpha (CNTFRα), a cDNA insert encoding cardiotrophin-like cytokine factor 1 (CLC), a cDNA insert encoding cytokine receptor-like factor 1 (CLF), and a cDNA insert encoding a CNTFRα-CLC fusion protein.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided in this document.

FIG. 1 shows AAV-derived muscle CNTFRα protein translocates to a widespread population of motor neurons. Hindlimb (gastrocnemius and soleus) muscles of SOD1^{G93A} mice were injected at 120 d with vehicle or AAV1.1-CNTFRα (3×10¹⁰vg per hindlimb) modified to produce HA-tagged CNTFRα protein, which was immunohistochemically localized 1 month post-injection (n=3/group). Images show vector-derived CNTFRα levels in: (A) gastrocnemius of AAV1.1-CNTFRα-treated mice; (B) gastrocnemius of control mice; (C) triceps brachii forelimb muscle; (D, F, H, I, J) spinal cord motor neuron (MN) soma of AAV1.1-CNTFRα-treated mice (denoted by arrows); (E, G, K) spinal cord MN soma of control mice; (L) sera, denaturing Western blot; mature glycosylated CNTFRα runs at ˜60 Kda); (M, N, O) clusters of rostral spinal cord putative ventral root axons of AAV1.1-CNTFRα-treated mice; (P) clusters of rostral spinal cord putative ventral root axons of control mice. Scale bars: A-C: 100 um; D-K: 25 um: L-O: 25 um.

FIG. 2 shows reduced MN terminal loss in AAV1.1-CNTFRα-treated SOD1^{G93A} mice. AAV1.1-CNTFRα (3×10¹⁰vg per hindlimb) or empty vector control was injected into SOD1^{G93A} mouse hindlimb muscles at 120 days. (A) YFP16 labeled MN axons and terminals in control mice; (B) α-bungarotoxin labeled post-synaptic endplates in control mice; (C) merged image of (A) and (B); (D) YFP16 labeled MN axons and terminals in AAV1.1-CNTFRα-treated mice; (E) α-bungarotoxin labeled post-synaptic endplates in AAV1.1-CNTFRα-treated mice; (F) merged image of (D) and (E). Innervated endplates=white arrows; non-innervated endplates=grey arrows. Scale bar=50 μm.

FIG. 3 shows AAV1.1-CNTFRα treatment inhibits ALS in SOD1^{G93A} mice. AAV1.1-CNTFRα (3×10¹⁰vg) or vehicle was injected bilaterally into gastrocnemius and soleus muscles of randomly assigned SOD1^{G93A} littermates at 120 days of age. AAV1.1-CNTFRα delayed end stage paralysis. 2-tailed log rank p value shown (AAV1.1-CNTFRα, 173.0±3.7d, n=14; controls run in parallel, mean=161.1±2.0d, n=26, p=0.0033).

FIG. 4 shows AAV1.1-CNTFRα treatment slows ALS motor decline in SOD1G93A mice. AAV1.1-CNTFRα slowed decline in motor function accessed by hind limb grip strength (top panel) and rotarod performance (bottom panel). Injection time indicated by arrows. ANOVA p values (p<0.0001) shown for main effect of treatment (control, n=15; AAV1.1-CNTFRα, n=14).

FIG. 5 shows results of Western blot analysis of circulating (serum) CNTFRα levels after muscle-specific CNTFRα depletion.

FIG. 6 shows AAV1.1-CLC treatment inhibits ALS in SOD1^{G93A} mice. AAV1.1-CLC (3×10¹⁰vg) or vehicle was injected bilaterally into gastrocnemius and soleus muscles of randomly assigned SOD1^{G93A} littermates at 120 days of age. AAV1.1-CLC treatment delayed end stage paralysis. 2-tailed log rank p value shown (AAV1.1-CLC, n=17; controls run in parallel, n=26).

FIG. 7 shows combined AAV1.1-CNTFRα and AAV1.1-CLC treatment inhibits ALS in SOD mice. Combined treatment or vehicle was injected bilaterally into gastrocnemius and soleus muscles of randomly assigned female SOD1^{G93A} littermates at 120 days of age. The treatment delayed end stage paralysis. 2-tailed log rank p value shown (Combined AAV treatment, n=10; controls run in parallel, n=26).

FIG. 8 shows muscle-specific CNTFRα knock-down accelerates SOD1^{G93A} disease. Muscle CNTFRα mlclf-Cre knockdown (n=8) and littermate controls (n=18) were monitored as described herein. Knockdown mice reached end stage paralysis significantly earlier. 2-tailed log rank p value shown.

FIG. 9 shows muscle-specific CNTFRα knockdown greatly accelerates the final phase of SOD1^{G37R} disease. (A) Muscle-specific CNTFRα knockdown with mlclf-Cre (n=10 control, 14 knockdown); and (B) Adult onset (2 month) muscle-specific CNTFRα knock-down with HSA-MCM-Cre (n=9 control, 10 knock-down) both greatly accelerated SOD1^{G37R} paralysis onset to end stage (2-tailed log rank p values shown).

FIG. 10 shows muscle-specific CNTFRα knockdown accelerates TDP-43^{Q331K} disease. Muscle-specific (HSA-MCM-Cre) CNTFRα knockdown and control mice (all TDP-43^{Q331K}) were monitored 2×/wk for TDP-43^{Q331K}-induced hindlimb clasp motor deficit. Lines correspond to individual mice, depicting hindlimb clasp history and age. The knockdown greatly accelerated hindlimb clasp onset (2-tailed log rank; p=0.0008). Muscle CNTFRα knockdown does not produce hindlimb clasp in naïve mice, so the knockdown accelerated TDP-43^{Q331K} disease and muscle CNTFRα protects against this ALS-inducing mutation. Two of the knockdown mice reached end stage (indicated by X).

FIG. 11 shows AAV1.1-BDNF and AAV1.1-NT-3 treatments led to increased survival in AAV1.1-CLC-treated SOD1^{G93A} ALS mice.

FIG. 12 shows AAV1.1-BDNF and AAV1.1-NT-3 treatments led to increased survival in AAV1.1-CNTFRα-treated SOD1^{G93A} ALS mice and AAV1.1-BDNF-treatment led to increased survival in SOD1^{G93A} ALS mice receiving no other treatment.

### SEQUENCE LISTING

The nucleic and/or amino acid sequences listed herein are shown using standard letter abbreviations for nucleotide bases or amino acids as defined in 37 C.F.R. 1.822. Only one strand of each nucleic acid sequence is shown, but the complementary strand is understood as included by any reference to the displayed strand. In the accompanying sequence listing:

SEQ ID NO: 1 represents a mature human CNTFRα protein sequence.

SEQ ID NO: 2 represents a human CNTFRα protein sequence encoded by an AAV vector.

SEQ ID NO: 3 represents a mature mouse CNTFRα protein sequence.

SEQ ID NO: 4 represents a mouse CNTFRα protein sequence encoded by an AAV vector.

SEQ ID NO: 5 represents a mature human CLC protein sequence.

SEQ ID NO: 6 represents a human CLC protein sequence encoded by an AAV vector.

SEQ ID NO: 7 represents a mature mouse CLC protein sequence.

SEQ ID NO: 8 represents a mouse CLC protein sequence encoded by an AAV vector.

SEQ ID NO: 9 represents a mature human CLF protein sequence.

SEQ ID NO: 10 represents a human CLF protein sequence encoded by an AAV vector.

SEQ ID NO: 11 represents a mature mouse CLF protein sequence.

SEQ ID NO: 12 represents a mouse CLF protein sequence encoded by an AAV vector.

SEQ ID NO: 13 represents a human mature BDNF protein sequence.

SEQ ID NO: 14 represents a human BDNF protein sequence encoded by an AAV vector.

SEQ ID NO: 15 represents a mature mouse BDNF protein sequence.

SEQ ID NO: 16 represents a mouse BDNF protein sequence encoded by an AAV vector.

SEQ ID NO: 17 represents a mature human NT-3 protein sequence.

SEQ ID NO: 18 represents a human NT-3 protein sequence encoded by an AAV vector.

SEQ ID NO: 19 represents a mature mouse NT-3 protein sequence.

SEQ ID NO: 20 represents a mouse NT-3 protein sequence encoded by an AAV vector.

SEQ ID NO: 21 represents a mature human CNTFRα-CLC fusion protein suitable for IV administration.

SEQ ID NO: 22 represents a human CNTFRα-CLC fusion protein encoded by an AAV vector.

SEQ ID NO: 23 represents an exemplary linker polypeptide sequence.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

The practice of the present invention will employ, unless otherwise indicated, conventional methods of virology, microbiology, molecular biology and recombinant DNA techniques within the skill of the art. Such techniques are explained fully in the literature. See, e.g., Sambrook et al. Molecular Cloning: A Laboratory Manual (Current Edition); DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (B. N. Fields and D. M. Knipe, eds.); Freshney Culture of Animal Cells, A Manual of Basic Technique (Wiley-Liss, Third Edition); and Ausubel et al. (1991) Current Protocols in Molecular Biology (Wiley Interscience, NY).

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

“Functionally equivalent,” as used herein, refers to a CNTFRα, CLC, CLF, BDNF, or NT-3 polypeptide that retains some or all of the biological properties regarding inhibition of motor neuron degenerative disorders, such as ALS, but not necessarily to the same degree, as a native CNTFRα, CLC, CLF, BDNF, or NT-3 molecule. In some embodiments, the fragments of CNTFRα, CLC, CLF, BDNF, or NT-3 proteins comprise shorter polypeptides derived from the full length CNTFRα, CLC, CLF, BDNF, or NT-3 proteins, which are functionally equivalent.

“Homology” refers to the percent similarity between two polynucleotide or two polypeptide moieties. Two polynucleotide, or two polypeptide sequences are “substantially homologous” to each other when the sequences exhibit at least about 50%, at least about 75%, at least about 80%-85%, at least about 90%, or at least about 95%-99% or more sequence similarity or sequence identity over a defined length of the molecules. As used herein, “substantially homologous” also refers to sequences showing complete identity to the specified polynucleotide or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

The term “variant” refers to a biologically active derivative of the reference molecule, or a fragment of such a derivative, that retains desired activity, such as anti-ALS activity in the assays described herein. In general, the term “variant” refers to compounds having a native polypeptide sequence and structure with one or more amino acid additions, substitutions (generally conservative in nature) and/or deletions, relative to the native molecule, so long as the modifications do not destroy anti-ALS activity. In embodiments, the variant has at least the same biological activity as the native molecule.

As used herein, the term “motor neuron degenerative disorder” refers to a degenerative disorder affecting a neuron with motor function. Motor neuron degenerative disorders include, but are not limited to, amyotrophic lateral sclerosis (ALS), primary lateral sclerosis (PLS), progressive muscular atrophy (PMA), progressive bulbar palsy (PBP), pseudobulbar palsy, peripheral neuropathy, spinal muscular atrophy, and Kennedy's disease. Motor neuron degenerative disorders cause increasing disability and can be terminal in nature. In a particular embodiment, the motor neuron degenerative disorder treated by the compositions and methods disclosed herein is ALS.

ALS is characterized by stages progressing in severity. In early stage ALS disease, muscles may be weak and soft or stiff, tight, and spastic. Muscle cramping and twitching occurs, as does loss of muscle bulk. Symptoms may be limited to a single body region or mild symptoms may affect more than one region. The subject suffering from ALS may experience fatigue, poor balance, slurred words, weak grip, tripping, or other minor symptoms. Middle stage ALS is characterized by more widespread symptoms, muscle paralysis, or muscle weakening. Cramping and twitching may also be present. Unused muscles may cause contractures, whereby joints may become rigid, painful, and deformed. Weakness in swallowing muscles may cause choking and difficulties eating. Weakened breathing muscles can lead to respiratory insufficiency, particularly when lying prone. Subjects may also experience inappropriate laughing or crying (pseudobulbar affect). In late stage ALS, most voluntary muscles are paralyzed. Respiratory muscles are severely compromised. Mobility is limited and assistance is required for personal care. Poor respiration may cause fatigue, confusion, headaches, and pneumonia. Speech, eating, and drinking may not be possible. In certain embodiments, the methods and compositions described herein are useful in treating a subject suffering from ALS. In a specific embodiment, the methods and compositions are useful in treating subjects suffering from early, middle, or late stage ALS. In a very specific embodiment, the methods and compositions are useful in treating late stage ALS disease.

In embodiments, a subject is considered to be suffering from early stage ALS when the subject experiences clinical symptoms in one central nervous system (CNS) region of the body. In embodiments, said “regions” are selected from the group consisting of bulbar, upper limb, lower limb, and diaphragmatic CNS regions. The bulbar region includes muscles of the mouth or throat of the subject. The upper limb region includes the hands, arms, axilla, and shoulders of the subject. The lower limb region includes the thighs, legs, and feet of the subject. The diaphragmatic region includes the respiratory (inspiratory and expiratory) muscles, such as the diaphragm, intercostals, and the like.

In embodiments, a subject is considered to be suffering from middle stage ALS when the subject experiences clinical symptoms in two CNS regions of the body.

In embodiments, a subject is considered to be suffering from late stage ALS when the subject requires a gastrostomy, requires non-invasive ventilation, and/or experiences clinical symptoms in at least three CNS regions of the body.

In another embodiment, the compositions and methods described herein are suitable for use and effective after symptom onset, and more specifically, after symptom onset and diagnosis.

Studies have shown that a subset of patients with ALS possesses mutations in certain genes. One such mutation occurs in the TDP-43 (TARDBP) gene, which suggests that gain of toxic function or loss of function in TDP-43 may be an underlying cause of ALS. Pesiridis, et al., Mutations in TDP-43 link glycine-rich domain functions to amyotrophic lateral sclerosis, Human Mol. Genet. 18(R2): R156-R162 (2009). In certain embodiments, the methods and compositions disclosed herein are useful for treating ALS characterized by one or both of at least one TDP-43 mutation and abnormal TDP-43 distribution in a subject.

As used herein, the term “subject” refers to any mammalian subject, including humans, non-human primates, pigs, dogs, rats, mice, and the like. In a specific embodiment, the subject is a human.

An “effective amount” is an amount sufficient to achieve beneficial or desired results. An effective amount can be administered in one or more administrations, applications or dosages. The effective amount of the proteins, fusion proteins, or modified AAV vectors, for use in the pharmaceutical compositions and methods herein will vary with the motor neuron degenerative disorder being treated, the age and physical condition of the subject to be treated, the severity of the condition, the duration of the treatment, the nature of concurrent therapy, the particular proteins, fusion proteins, or modified AAV vectors being employed, the particular pharmaceutically-acceptable carriers utilized, and like factors within the knowledge and expertise of the attending physician.

Brain derived neurotrophic factor (BDNF) is a neurotrophic factor that supports differentiation, maturation, and survival of neurons in the nervous system. BDNF has been shown to elicit a neuroprotective effect under adverse conditions. Bathina, et al., Brain-derived neurotrophic factor and its clinical implications, Arch. Med. Sci. 11(6): 1164-78 (2015). A number of BDNF polynucleotide and amino acid sequences are known. Suitable amino acid sequences of human and mouse BDNF are set forth herein as SEQ ID NOs: 13-16. Additional protein coding BDNF sequences are known in the art. Any of these sequences, as well as variants thereof, such as sequences substantially homologous and functionally equivalent to these sequences, will find use in the present methods. In embodiments, the BDNF protein shares at least 80%, at least 85%, at least 90%, at least 95%, at least 96%, at least 97%, at least 98%, at least 99%, or 100% identity with any of SEQ ID NOs: 13-16 or any subset thereof.

Neurotrophin-3 (NT-3) is a neurotrophic factor in the nerve growth factor (NGF) family with activity on certain neurons of the peripheral and central nervous system. Mice born without the ability to make NT-3 have loss of motor neuron axonal terminals. A number of NT-3 polynucleotide and amino acid sequences are known. Representative protein coding sequences of human and mouse NT-3 are set forth herein as SEQ ID NOs: 17-20. Additional NT-3 sequences are known in the art. Any of these sequences, as well as variants thereof, such as sequences substantially homologous and functionally equivalent to these sequences, will find use in the present methods. In embodiments, the NT-3 protein shares at least 80%, at least 85%, at least 90%, at least 95%, at least 96%, at least 97%, at least 98%, at least 99%, or 100% identity with any of SEQ ID NOs: 17-20 or any subset thereof.

Ciliary neurotrophic factor receptor alpha (CNTFRα) is an essential ligand binding subunit of the CNTF receptor, which is composed of CNTFRα, a leukemia inhibitory factor receptor β (LIFRβ), and glycoprotein (gp) 130. While LIFRβ and gp130 are found in other related receptors, CNTFRα is unique to CNTF receptors and is required for all known forms of CNTF receptor signaling. A number of CNTFRα polynucleotide and amino acid sequences are known. The degree of homology between rat, human, and mouse proteins is about 94%. Representative amino acid sequences of human and mouse CNTFRα are set forth herein as SEQ ID NOs: 1-4. Additional mammalian protein coding CNTFRα sequences are known in the art. Any of these sequences, as well as variants thereof, such as sequences substantially homologous and functionally equivalent to these sequences, will find use in the present methods. In embodiments, the CNTFRα protein shares at least 80%, at least 85%, at least 90%, at least 95%, at least 96%, at least 97%, at least 98%, at least 99%, or 100% identity with any of SEQ ID NOs: 1-4 or any subset thereof.

Cardiotrophin-like cytokine factor 1 (CLC) is a member of the gp130 cytokine family and is also referred to as CLCF-1, novel neurotrophin-1 (NNT-1), or B cell-stimulating factor-3 (BSF-3). CLC forms a heterodimer complex with cytokine receptor-like factor 1 (CLF). This dimer competes with ciliary neurotrophic factor (CNTF) for binding to the ciliary neurotrophic factor receptor, and activates the Jak-STAT signaling cascade. CLC can be actively secreted from cells by forming a complex with soluble type I CLF or soluble CNTFRα. The CLC/CNTFRα complex also activates CNTF receptors.

CLC is a potent neurotrophic factor, B-cell stimulatory agent and neuroendocrine modulator of pituitary corticotroph function. A number of CLC polynucleotide and amino acid sequences are known. Representative amino acid sequences of human and mouse CLC are set forth herein as SEQ ID NOs: 5-8. Additional protein coding CLC sequences are known in the art. See, e.g., NCBI accession numbers AK298052, BC012939, DC393345, and BM846622 (human); AC109138, AI451696, AK137396, BB786146, BC104258, and CX202966 (mouse); and BC098643 (rat) (all accession numbers accessed Sep. 3, 2020, 12:00 a.m. EST); as well as other mammalian CLC sequences. Any of these sequences, as well as variants thereof, such as sequences substantially homologous and functionally equivalent to these sequences, will find use in the present methods. In embodiments, the CLC protein shares at least 80%, at least 85%, at least 90%, at least 95%, at least 96%, at least 97%, at least 98%, at least 99%, or 100% identity with any of SEQ ID NOs: 5-8 or any subset thereof.

Cytokine receptor-like factor 1 (CLF) is a member of the cytokine type I receptor family. The protein forms a secreted complex with cardiotrophin-like cytokine factor 1 (CLC) and acts on cells expressing ciliary neurotrophic factor receptors. The complex can promote survival of neuronal cells. A number of CLF polynucleotide and amino acid sequences are known. Representative amino acid sequences of human and mouse CLF are set forth herein as SEQ ID NOs: 9-12. Additional protein coding CLF sequences are known in the art. See, e.g., NCBI accession numbers AF073515 and AY358291 (human); AC157774 (mouse); CH474031 (rat); and BC076526 (zebrafish) (all accession numbers accessed Sep. 3, 2020, 12:00 a.m. EST); as well as other mammalian CLF sequences. Any of these sequences, as well as variants thereof, such as sequences substantially homologous and functionally equivalent to these sequences, will find use in the present methods. In embodiments, the CLF protein shares at least 80%, at least 85%, at least 90%, at least 95%, at least 96%, at least 97%, at least 98%, at least 99%, or 100% identity with any of SEQ ID NOs: 9-12 or any subset thereof.

A CNTFRα-CLC fusion protein comprises a CNTFRα protein or fragment thereof covalently linked to a CLC protein or fragment thereof. A CNTFRα-CLC fusion protein suitable for use in the instant methods is described in Guillet, et al., Functionally active fusion protein of the novel composite cytokine CLC/soluble CNTF receptor, Eur. J. Biochem. 269: 1932-41 (2002), incorporated herein by reference in its entirety. In embodiments, the CNTFRα-CLC fusion protein shares at least 80%, at least 85%, at least 90%, at least 95%, at least 96%, at least 97%, at least 98%, at least 99%, or 100% identity with SEQ ID NO: 21 or SEQ ID NO: 22.

The disclosed fusion proteins optionally comprises a polypeptide linker between the CNTFRα and CLC constructs. For example, amino acids 324-337 of SEQ ID NO: 21, or amino acids 337-350 of SEQ ID NO: 22 correspond to linkers having the amino acid sequence LEGGGGSGGGGSLE (SEQ ID NO: 23). However, the skilled artisan will appreciate that other flexible polypeptide linkers are also suitable for use in the disclosed fusion proteins. Exemplary suitable linkers are disclosed, for example, in Chen, et al., Fusion Protein Linkers: Property, Design and Functionality, Adv. Drug. Deliv. Rev. 65(10): 1357-69 (2013), incorporated herein by reference in its entirety.

### AAV Vectors Comprising Recombinant Genomes

Adeno-associated virus (AAV) is a small, nonenveloped icosahedral virus that infects humans and other primates, but causes only weak immune response and is not considered pathogenic. At present, multiple AAV serotypes (AAV1-AAV13) have been sequenced and studies have identified other AAV genomes. AAVs differ in tropism for target tissues, including cardiac and skeletal muscle, liver and lung tissue, and cells in the CNS. Differing tropisms can be exploited for use in gene therapy, enabling the directed treatment of specific tissues. Drouin, et al., Adeno-associated virus structural biology as a tool in vector development, Future. Virol. 8(12): 1183-99 (2013). AAV vectors modified to be muscle-tropic are particularly useful in the presently disclosed methods. By an “AAV vector” is meant a vector derived from an adeno-associated virus serotype.

Disclosed herein are modified AAV vectors engineered to comprise a recombinant AAV-based genome (rAAV). rAAV genomes can be single stranded rAAV, which requires synthesis of a complementary DNA strand, or self-complementary rAAV (scrAAV), which comprises two shorter DNA strands that are complementary to each other. By avoiding second-strand synthesis, scrAAV can express more quickly, although vector capacity is reduced. In certain embodiments, the AAV vectors disclosed herein comprise an rAAV genome comprising single stranded rAAV, self-complementary rAAV (scrAAV), and combinations thereof.

Each therapeutic vector disclosed herein comprises an rAAV genome engineered to comprise at least one cDNA insert protein coding sequence. In one embodiment, the cDNA insert comprises one or more of a BDNF-encoding cDNA insert, or an NT-3-encoding cDNA insert. In certain embodiments, the cDNA insert is a BDNF-encoding cDNA insert. In another embodiment, the cDNA insert is an NT-3-encoding cDNA insert. In another embodiment, the rAAV genome may comprise both a BDNF-encoding cDNA insert and an NT-3-encoding cDNA insert. While not desiring to be bound by theory, it is believed that the administration of AAV vectors comprising rAAV genomes comprising BDNF- and/or NT-3-encoding cDNA inserts to a subject suffering from a motor neuron degenerative disorder is therapeutic by enhancing the effects of endogenous mechanisms that protect motor neurons, including those mechanisms that involve CNTF receptors. Further, it is believed that the motor neuron protective CNTF receptor mechanisms involve the systemic release of CNTFRα expressed by skeletal muscle.

In embodiments, the BDNF-encoding cDNA insert encodes a protein having at least 80%, at least 85%, at least 90%, at least 95%, at least 96%, at least 97%, at least 98%, at least 99%, or 100% identity with SEQ ID NO: 14 or SEQ ID NO: 16.

In embodiments, the NT-3-encoding cDNA insert encodes a protein having at least 80%, at least 85%, at least 90%, at least 95%, at least 96%, at least 97%, at least 98%, at least 99%, or 100% identity with SEQ ID NO: 18 or SEQ ID NO: 20.

The AAV vectors disclosed herein comprise control elements capable of directing the in vivo transcription and translation of BDNF or NT-3. In certain embodiments, the control elements comprise a promoter. The term “promoter” is used herein to refer to a nucleotide region comprising a DNA regulatory sequence, wherein the regulatory sequence is derived from a gene which is capable of binding RNA polymerase and initiating transcription of a downstream (3′-direction) coding sequence. In one embodiment, the promoter is selected from a cytomegalovirus early enhancer element/chicken beta-actin (CAG) promoter and a muscle-specific promoter. In a specific embodiment, the muscle-specific promoter is a muscle specific creatine kinase (MCK) promoter. In a more specific embodiment, the MCK promoter comprises double muscle specific creatine kinase (dMCK) or triple muscle specific creatine kinase (tMCK) promoter. The skilled artisan will appreciate that other promoters are known in the art and suitable for use in controlling and directing the in vivo transcription and translation of BDNF and/or NT-3.

In embodiments, the AAV vectors comprising a BDNF-encoding cDNA insert and/or an NT-3-encoding cDNA insert may be administered in combination with one or more additional AAV vectors comprising a cDNA insert selected from the group consisting of a ciliary neurotrophic factor receptor alpha (CNTFRα)-encoding cDNA insert, a cardiotrophin-like cytokine (CLC)-encoding cDNA insert, a cytokine receptor-like factor 1 (CLF)-encoding cDNA insert, or a CNTFRα-CLC fusion protein-encoding cDNA insert.

In another embodiment, a modified adeno-associated virus (AAV) vector comprising a plurality of recombinant AAV (rAAV)-based genomes is provided, wherein the rAAV genomes comprise at least one of: a BDNF-encoding cDNA insert or an NT-3-encoding cDNA insert; and one or more of a ciliary neurotrophic factor receptor alpha (CNTFRα)-encoding cDNA insert, a cardiotrophin-like cytokine factor 1 (CLC)-encoding cDNA insert, a cytokine receptor-like factor 1 (CLF)-encoding cDNA insert, and a CNTFRα-CLC fusion protein-encoding cDNA insert.

In embodiments, the CNTFRα-encoding cDNA insert encodes a protein having at least 80%, at least 85%, at least 90%, at least 95%, at least 96%, at least 97%, at least 98%, at least 99%, or 100% identity with SEQ ID NO: 2 or SEQ ID NO: 4.

In embodiments, the CLC-encoding cDNA insert encodes a protein having at least 80%, at least 85%, at least 90%, at least 95%, at least 96%, at least 97%, at least 98%, at least 99%, or 100% identity with SEQ ID NO: 6 or SEQ ID NO: 8.

In embodiments, the CLF-encoding cDNA insert encodes a protein having at least 80%, at least 85%, at least 90%, at least 95%, at least 96%, at least 97%, at least 98%, at least 99%, or 100% identity with SEQ ID NO: 10 or SEQ ID NO: 12.

In embodiments, the CNTFRα-CLC fusion protein-encoding cDNA insert encodes a protein having at least 80%, at least 85%, at least 90%, at least 95%, at least 96%, at least 97%, at least 98%, at least 99%, or 100% identity with SEQ ID NO: 21 or SEQ ID NO: 22.

### Methods of Treatment

In one embodiment, a method of treating a subject suffering from a motor neuron degenerative disorder is provided, the method comprising administering to the subject one or more modified adeno-associated virus (AAV) vectors comprising a recombinant AAV (rAAV)-based genome, wherein the rAAV-based genome comprises one or more of: a brain derived neurotrophic factor (BDNF)-encoding cDNA insert or a neurotrophin-3 (NT-3)-encoding cDNA insert.

In embodiments, the BDNF-encoding cDNA insert encodes a protein having at least 80%, at least 85%, at least 90%, at least 95%, at least 96%, at least 97%, at least 98%, at least 99%, or 100% identity with SEQ ID NO: 14 or SEQ ID NO: 16.

In embodiments, the NT-3-encoding cDNA insert encodes a protein having at least 80%, at least 85%, at least 90%, at least 95%, at least 96%, at least 97%, at least 98%, at least 99%, or 100% identity with SEQ ID NO: 18 or SEQ ID NO: 20.

In embodiments, methods of treatment further comprise administering to the subject one or more additional modified AAV vectors comprising an rAAV-based genome, wherein the rAAV-based genome comprises one or more of: a ciliary neurotrophic factor receptor alpha (CNTFRα)-encoding cDNA insert, a cardiotrophin-like cytokine (CLC)-encoding cDNA insert, a cytokine receptor-like factor 1 (CLF)-encoding cDNA insert, or a CNTFRα-CLC fusion protein-encoding cDNA insert.

In embodiments, the CNTFRα-encoding cDNA insert encodes a protein having at least 80%, at least 85%, at least 90%, at least 95%, at least 96%, at least 97%, at least 98%, at least 99%, or 100% identity with SEQ ID NO: 2 or SEQ ID NO: 4.

In embodiments, the CLC-encoding cDNA insert encodes a protein having at least 80%, at least 85%, at least 90%, at least 95%, at least 96%, at least 97%, at least 98%, at least 99%, or 100% identity with SEQ ID NO: 6 or SEQ ID NO: 8.

In embodiments, the CLF-encoding cDNA insert encodes a protein having at least 80%, at least 85%, at least 90%, at least 95%, at least 96%, at least 97%, at least 98%, at least 99%, or 100% identity with SEQ ID NO: 10 or SEQ ID NO: 12.

In embodiments, the CNTFRα-CLC fusion protein-encoding cDNA insert encodes a protein having at least 80%, at least 85%, at least 90%, at least 95%, at least 96%, at least 97%, at least 98%, at least 99%, or 100% identity with SEQ ID NO: 21 or SEQ ID NO: 22.

In one embodiment, a method of treating a subject suffering from a motor neuron degenerative disorder is provided, the method comprising administering to the subject an effective amount of a modified adeno-associated virus (AAV) vector comprising a plurality of recombinant AAV (rAAV)-based genomes, wherein the plurality of rAAV genomes comprises at least one of: a BDNF-encoding cDNA insert or an NT-3-encoding cDNA insert; and one or more of a ciliary neurotrophic factor receptor alpha (CNTFRα)-encoding cDNA insert, a cardiotrophin-like cytokine factor 1 (CLC)-encoding cDNA insert, a cytokine receptor-like factor 1 (CLF)-encoding cDNA insert, and a CNTFRα-CLC fusion protein-encoding cDNA insert.

In another embodiment, a method for treating a subject suffering from a motor neuron degenerative disorder is provided, the method comprising: administering to the subject an effective amount of a brain derived neurotrophic factor (BDNF) protein or fragment thereof and/or an NT-3 protein or fragment thereof. In embodiments, the BDNF protein shares at least 80%, at least 85%, at least 90%, at least 95%, at least 96%, at least 97%, at least 98%, at least 99%, or 100% identity with SEQ ID NO: 13 or SEQ ID NO: 15. In embodiments, the NT-3 protein shares at least 80%, at least 85%, at least 90%, at least 95%, at least 96%, at least 97%, at least 98%, at least 99%, or 100% identity with SEQ ID NO: 17 or SEQ ID NO: 19.

In some embodiments, the method further comprises administering to the subject an effective amount of one or more of: a CNTFRα protein or fragment thereof; a CLC protein or fragment thereof; a CLF protein or fragment thereof; and a CNTFRα-CLC fusion protein.

In embodiments, the CNTFRα protein shares at least 80%, at least 85%, at least 90%, at least 95%, at least 96%, at least 97%, at least 98%, at least 99%, or 100% identity with SEQ ID NO: 1 or SEQ ID NO: 3.

In embodiments, the CLC protein shares at least 80%, at least 85%, at least 90%, at least 95%, at least 96%, at least 97%, at least 98%, at least 99%, or 100% identity with SEQ ID NO: 5 or SEQ ID NO: 7.

In embodiments, the CLF protein shares at least 80%, at least 85%, at least 90%, at least 95%, at least 96%, at least 97%, at least 98%, at least 99%, or 100% identity with SEQ ID NO: 9 or SEQ ID NO: 11.

A CNTFRα-CLC fusion protein comprises a CNTFRα protein or fragment thereof covalently linked to a CLC protein or fragment thereof. A CNTFRα-CLC fusion protein suitable for use in the instant methods is described in Guillet, et al., Functionally active fusion protein of the novel composite cytokine CLC/soluble CNTF receptor, Eur. J. Biochem. 269: 1932-41 (2002), incorporated herein by reference in its entirety. In embodiments, the CNTFRα-CLC fusion protein shares at least 80%, at least 85%, at least 90%, at least 95%, at least 96%, at least 97%, at least 98%, at least 99%, or 100% identity with SEQ ID NO: 21 or SEQ ID NO: 22. In embodiments, the CNTFRα-CLC fusion protein having SEQ ID NO: 21 may be administered systemically to a subject. In embodiments, the CNTFRα-CLC fusion protein of SEQ ID NO: 22 may be included in an AAV vector for non-systemic administration to a subject.

In embodiments, the proteins or fragments thereof are administered sequentially or concurrently. In embodiments, at least one of the proteins or fragments thereof administered in the combination is BDNF, NT-3, or a CNTFRα-CLC fusion protein.

In embodiments, the motor neuron degenerative disorder treated in any of the methods disclosed herein is selected from the group consisting of amyotrophic lateral sclerosis (ALS), primary lateral sclerosis (PLS), progressive muscular atrophy (PMA), progressive bulbar palsy (PBP), pseudobulbar palsy, peripheral neuropathy, spinal muscular atrophy, Kennedy's disease, and spinal muscle atrophy (SMA). In a specific embodiment, the disorder is ALS. In a very specific embodiment, the ALS is late-stage ALS. In embodiments, the motor neuron degenerative disorder is ALS characterized by one or both of: at least one TDP-43 mutation; and an abnormal cellular TDP-43 distribution.

In embodiments, the methods of treatment disclosed herein comprise intravenous injection of the proteins, fusion proteins, or modified AAV vectors described herein. In embodiments, the methods of treatment disclosed herein comprise non-systemic administration of the disclosed proteins, fusion proteins or AAV vectors. In a specific example, administration of the modified AAV vectors disclosed herein is non-systemic administration, more specifically intramuscular (IM) administration (injection). In a specific embodiment, the administration of the AAV vector comprises IM administration to one or more muscles selected from non-respiratory skeletal muscles, respiratory muscles, and combinations thereof.

In embodiments, treating the motor neuron degenerative disorder comprises inhibiting progression of the disorder in the subject in need thereof. In certain embodiments, administering the proteins, fusion proteins, or modified AAV vectors described herein is effective to at least temporarily reverse paralysis in the subject. In certain embodiments, administration of the proteins, fusion proteins, or modified AAV vectors described herein is effective to delay end stage paralysis or death in a subject suffering from ALS. In certain embodiments, administration of the proteins, fusion proteins, or modified AAV vectors described herein is effective to increase the amount of time from onset of paralysis to end stage paralysis or death in a subject suffering from ALS.

The step of administering the active agents described herein may be initiated prior to, contemporaneous with, or after an onset of clinical motor symptoms in the subject.

In embodiments, the methods described herein further comprise administering to the subject an effective amount of a docosahexanoic acid (DHA) dietary supplement. DHA is an omega-3 fatty acid known to have a positive effect on hypertension, arthritis, atherosclerosis, depression, adult-onset diabetes mellitus, myocardial infarction, thrombosis, and certain cancers. Further, DHA is required for development of the brain in infants and maintenance of normal brain function in healthy adults (Horrocks, et al., Health benefits of docosahexanoic acid (DHA), Pharmacol. Res. 40(3): 211-25 (1999)). DHA supplements are known in the art and are readily available from a variety of vendors (see, e.g., Nordic Naturals, Watsonville, Calif.).

### Pharmaceutical Compositions

In an embodiment, a pharmaceutical composition is provided, comprising: one or more muscle-tropic modified AAV vectors, each of said AAV vectors comprising an rAAV genome, each of said rAAV genomes engineered to comprise: (i) one or more of a BDNF-encoding cDNA insert or an NT-3-encoding cDNA insert; and (ii) a promoter; and at least one pharmaceutically-acceptable excipient.

In a specific embodiment, AAV vectors comprising a BDNF- and/or NT-3-encoding cDNA insert may be administered in combination with an AAV vector comprising a CNTFRα-encoding cDNA insert, a CLC-encoding cDNA insert, a CLF-encoding cDNA insert, and/or a CNTFRα-CLC fusion protein-encoding cDNA insert. In a more specific embodiment the AAV vectors comprising a BDNF- and/or NT-3-encoding cDNA insert are administered in combination with an AAV vector comprising a CLC-encoding cDNA insert. In another specific embodiment the AAV vectors comprising a BDNF- and/or NT-3-encoding cDNA insert are administered in combination with an AAV vector comprising a CNTFRα-encoding cDNA insert.

In another embodiment, a pharmaceutical composition is provided, comprising: a modified adeno-associated virus (AAV) vector comprising a plurality of recombinant AAV (rAAV)-based genomes, wherein the plurality of rAAV genomes comprises at least one of: a BDNF-encoding cDNA insert or an NT-3-encoding cDNA insert; and one or more of a ciliary neurotrophic factor receptor alpha (CNTFRα)-encoding cDNA insert, a cardiotrophin-like cytokine factor 1 (CLC)-encoding cDNA insert, a cytokine receptor-like factor 1 (CLF)-encoding cDNA insert, and a CNTFRα-CLC fusion protein-encoding cDNA insert; a promoter; and at least one pharmaceutically-acceptable excipient.

In embodiments, a pharmaceutical composition is provided, comprising an effective amount of a BDNF protein or fragment thereof; and at least one pharmaceutically-acceptable excipient. In a specific embodiment, the pharmaceutical composition is formulated for intravenous or intramuscular injection.

In embodiments, a pharmaceutical composition is provided, comprising an effective amount of an NT-3 protein or fragment thereof; and at least one pharmaceutically-acceptable excipient. In a specific embodiment, the pharmaceutical composition is formulated for intravenous or intramuscular injection.

In embodiments, a pharmaceutical composition is provided, comprising an effective amount of a fusion protein comprising a ciliary neurotrophic factor receptor alpha (CNTFRα) protein or fragment thereof covalently linked to a cardiotrophin-like cytokine (CLC) protein or fragment thereof; and at least one pharmaceutically-acceptable excipient. In a specific embodiment, the pharmaceutical composition is formulated for intravenous or intramuscular injection. Optionally, the CNTFRα-CLC fusion protein comprises a flexible polypeptide linker.

In embodiments, any of the BDNF, NT-3, or CNTFRα-CLC fusion proteins may be co-administered with an effective amount of a pharmaceutical composition comprising one or more of a CNTFRα protein or fragment thereof, a CLC protein or fragment thereof, or a CLF protein or fragment thereof.

Pharmaceutical compositions described herein may be formulated for intramuscular administration or intravenous administration.

Any two or more of the active agents, pharmaceutical compositions, and methods of treatment described herein (i.e., AAV vector compositions or intravenous or intramuscular injections comprising any of CNTFRα, CLC, CLF, BDNF, NT-3, and/or a CNTFRα-CLC fusion protein) may be administered to the subject in combination for enhanced therapeutic effect, in any permutation, as deemed appropriate by the skilled artisan. The effectiveness of such combination therapies may be further enhanced by administering to the subject an effective amount of a docosahexanoic acid (DHA) dietary supplement.

Active agents described herein can be co-administered (i.e., concurrently) or sequentially administered. When sequentially administered, the duration of time between administering a first active agent and administering a subsequent active agent may be from about 1 hour, 2 hours, 3 hours, 4 hours, 6 hours, 12 hours, 24 hours, one week, two weeks, three weeks, 4 weeks, one month, up to six months. In some embodiments, administration of one or more active agents as described herein is initiated prior to, contemporaneous with, or even after onset of motor symptoms in the subject. In a specific embodiment, administration is initiated during late stage ALS disease and is effective to slow disease progression and/or at least temporarily partially reverse motor symptoms in the subject, including paralysis. In another embodiment, administration is initiated after symptom onset, and more specifically after symptom onset and diagnosis.

Administration of active agents comprises systemic and non-systemic administration. In particular embodiments, non-systemic administration comprises intramuscular (IM) injection to one or more muscles selected from non-respiratory skeletal muscles, respiratory skeletal muscles, and combinations thereof. In certain embodiments, IM injections are administered unilaterally to a subject. In other embodiments, IM injections are administered bilaterally to the subject, for example, bilaterally to the gastrocnemius muscle on each side of the subject. When two or more active agents are administered, the two or more active agents can be independently administered; for example, one active agent may be administered systemically, and a second active agent may be administered non-systemically. Similarly, the location (i.e., skeletal muscle) and manner (i.e., unilateral or bilateral) of injection for non-systemic administration may vary between distinct active agents.

### EXAMPLES

The following examples are given by way of illustration and are not intended to limit the scope of the present disclosure.

### Example 1: AAV-Derived Muscle CNTFRα Protein Translocates to a Widespread Population of Motor Neurons

Hindlimb (gastrocnemius and soleus) muscles of SOD1^{G93A} mice were injected at 120 days with vehicle or AAV1.1-CNTFRα (3×10¹⁰vg per hindlimb) modified to produce HA-tagged CNTFRα protein, which was immunohistochemically localized 1 month post-injection (n=3/group).

qRT-PCR of end stage mice showed that the AAV1.1-CNTFRα treatment increases CNTFRα RNA in injected hindlimb gastrocnemius (GAS) muscle ˜5 fold (496±187% of vehicle SOD1^{G93A} controls; n=4) but does not affect lumbar spinal cord CNTFRα RNA (93±7% of same controls; n=4). Moreover, using primers specific for vector-derived mCNTFRα RNA (codon optimized mCNTFRα RNA differs from native RNA but encodes same amino acid) vector-derived RNA was readily found in all AAV1.1-mCNTFRα injected GAS muscles (1 month post-injection; n=3) but none was found in the lumbar spinal cords (at least 16,000 fold less than in GAS, given the PCR detection limit relative to GAS levels). Therefore, although some AAV vectors, under some conditions, transduce spinal MNs, IM injection of AAV1.1-CNTFRα clearly does not. Thus, the anti-ALS effect of AAV1.1-CNTFRα is not due to an increase in motor neuron (MN) CNTFRα RNA.

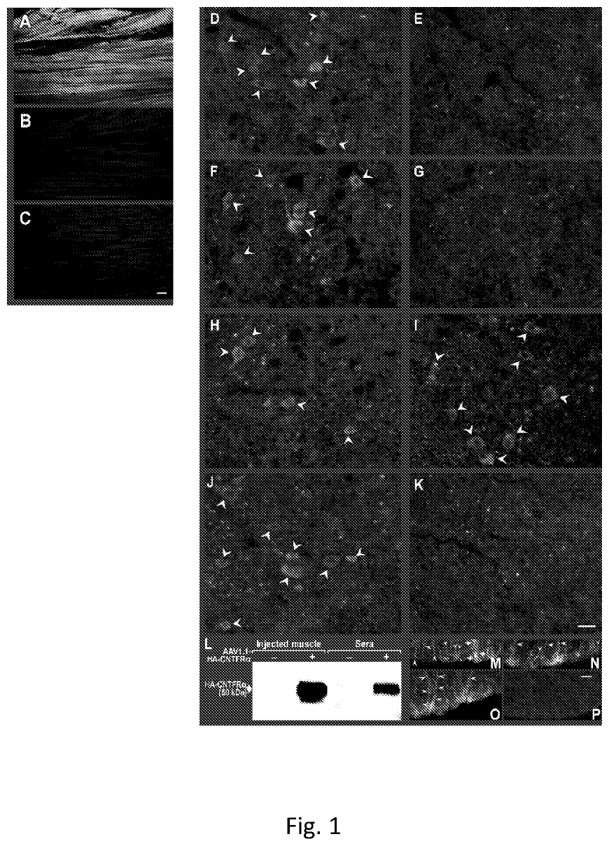

However, muscle CNTFRα protein is attached to the outer plasma membrane surface by a labile GPI link and is released in a soluble, functional form that may enhance neuroprotective CNTF receptors in MNs, if it translocates to MNs. The AAV1.1-CNTFRα was modified to produce HA-tagged mCNTFRα protein. SOD1^{G93A} mice were injected with this or vehicle at 120 days, and localized vector-derived CNTFRα protein (FIG. 1). As expected, high levels were observed in AAV1.1-CNTFRα injected GAS muscle (FIG. 1A) and none in controls (FIG. 1B). It was also found in many spinal cord MNs (FIG. 1D, F, H, I, J), and not in controls (FIG. 1E, G, K). Since MN CNTFRα RNA is not affected, the data suggest the vector-derived CNTFRα protein translocates from muscle to MNs. This was not limited to MNs innervating injected muscle, since vector-derived CNTFRα was also found in MNs throughout the thoracic cord (FIG. 1F, H, I, J; only other spinal cord region examined). These rostral MNs did not retrogradely transport the CNTFRα from muscles they innervate since those muscles did not contain vector-derived CNTFRα protein (e.g., FIG. 1C), indicating the hindlimb injected AAV1.1-CNTFRα does not transduce the rostral muscles. Confirming this, no vector-derived CNTFRα RNA was observed in the rostral muscles or rostral spinal cord.

Together, these data, along with the treatment's global therapeutic effect, suggest the vector-derived CNTFRα protein made in injected muscle is released globally/systemically and then translocates to and protects a widespread population of MNs, including MNs not innervating injected muscle. The suggestion that muscle CNTFRα can act on MNs not innervating the CNTFRα expressing muscle also agrees with the finding that muscle CNTFRα helps regenerate MN proximal axons at nerve lesions far from the CNTFRα expressing denervated muscle. While not desiring to be bound by theory, it is believed that this occurs through muscle CNTFRα being released systemically by denervation (be it lesion or ALS) and then globally taken up by MNs, since: 1) functional CNTFRα is released from denervated muscle; 2) this soluble CNTFRα is found in sera, CSF, and urine, and increased in human ALS; and 3) lesioned MN axons far from CNTFRα expressing denervated muscle display CNTF receptor dependent retrograde transport to MN soma.

The above treatment was also used to localize the vector-derived CNTFRα protein in sera by Western blot (FIG. 1L) and in lumbar and thoracic ventral root processes (FIG. 1M, N, O), consistent with a widespread population of MN axons taking up globally released CNTFRα and retrogradely transporting it to their soma. AAV and control mice were processed in parallel, including identical image capture and adjustment. Artifactual specks are seen with spinal cord sections from both groups but MN label is specific to AAV1.1-CNTFRα mice. The AAV1.1-CNTFRα treatment has a global protective effect on MN terminals (see Example 2, below).

Together, the data suggest the vector-derived CNTFRα protein is made in injected muscle, released systemically (including into sera), taken up by MNs (likely through axons) and protects their axon terminals from the ALS insult, thereby inhibiting disease progression.

### Example 2: Enhancing Muscle CNTFRα Expression Well after Symptom Onset Protects MN Terminals

To study AAV1.1-CNTFRα's global therapeutic effect, analysis focused on MN terminals in forelimb triceps brachii muscle because: animal and human data suggest, although many factors contribute to ALS MN degeneration, including glia, MN terminal loss likely underlies ALS symptoms and death, such that inhibiting this loss should be therapeutic; and standard AAV1.1-CNTFRα treatment (hindlimb muscle injection) does not affect CNTFRα levels in this forelimb muscle but increases CNTFRα in forelimb innervating MNs (Example 1) such that changes in triceps brachii MN terminals can serve as an anatomical index of AAV1.1-CNTFRα's global effects on MNs.

An empty vector (EV) control was prepared for use in the study. As expected, standard SOD1^{G93A} treatment protocol with the EV control yielded results equivalent to vehicle (EV mouse end stage=97.7±3.3% of vehicle injected littermates run in parallel; n=9 pair).

3×10vg of AAV1.1-CNTFRα or EV control was injected into SOD1^{G93A} mouse hindlimb muscles at 120 d. Analysis at any fixed age after the first mice are lost to end stage is confounded by group differences in subject loss. Analysis at end stage is confounded by group differences in end stage age. Thus, for a non-confounded comparison during the clinically important late stage disease, standard protocol was followed to inject one randomly selected member of each littermate pair with AAV1.1-CNTFRα and the other with EV control, and both mice were perfused together for analysis as soon as either reached end stage.

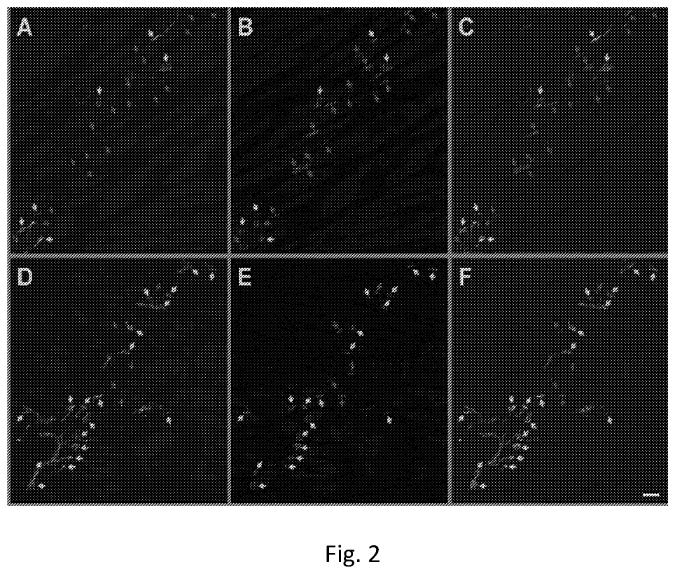

Results are shown in FIG. 2. Analysis of triceps brachii forelimb muscle MN terminals indicated more innervated endplates in AAV1.1-CNTFRα treated mice. YFP16 labeled MN axons and terminals (FIG. 2A, D), α-bungarotoxin labeled post-synaptic endplates (FIG. 2B, E), and merged images (FIG. 2C, F) of AAV1.1-CNTFRα (FIG. 2D-F) and control (FIG. 2A-C) treated littermates.

AAV1.1-CNTFRα mice had a 40.2% increase in fully innervated endplates per triceps brachii muscle (p=0.006; unpaired t-test; AAV1.1-CNTFRα=2726±96; control=1944±190; n=5 pair; FIG. 2) with no change in total endplates (p=0.393; AAV1.1-CNTFRα=8250±659; control=7586±327) or partially innervated endplates (p=0.934; AAV1.1-CNTFRα=542±124; control=556±109). The effect is particularly impressive considering triceps brachii was not injected or transduced, and therefore the increase in terminals reflects AAV1.1-CNTFRα's global effect on MNs. Further, treatment was well after symptom onset, so all MN degenerative processes (MN intrinsic and those involving other cells) were fully active until well after symptom onset, as in a typical ALS patient diagnosed and starting treatment after symptom onset.

### Example 3: AAV1.1-CNTFRα Treatment Inhibits ALS in SOD1G⁹³A Mice

ALS diagnosis takes approximately 1 year from symptom onset, so treatments must work when initiated well after symptom onset at diagnosis. Treating ALS mice earlier in disease has failed to identify human therapeutics, likely because inhibiting earlier disease processes does not predict effectiveness against late disease processes occurring at diagnosis. Clearly, there is a pressing need for treatments effective when started well after symptom onset.

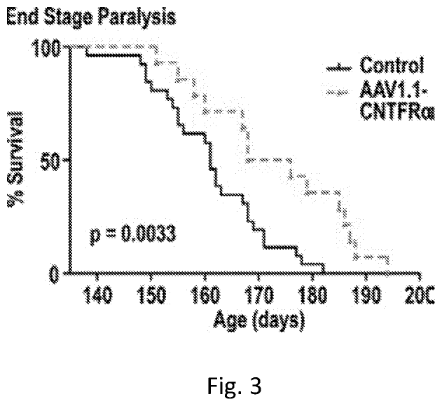

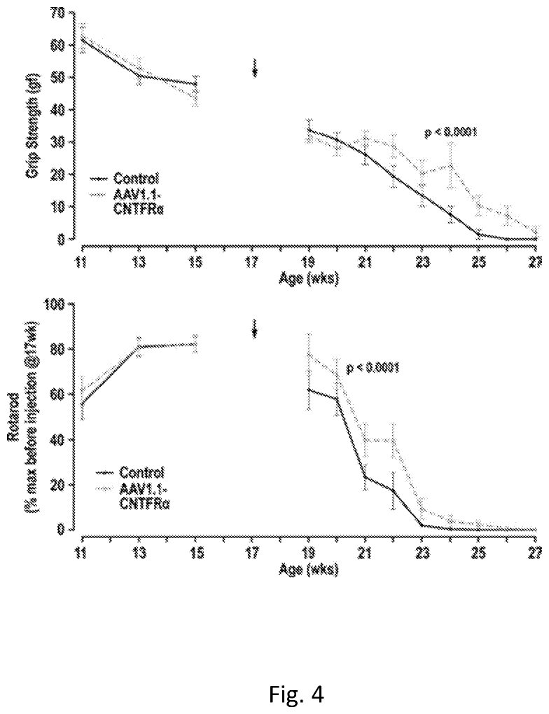

Safety and long-term expression make AAV the vector of choice for human gene therapy. AAV1 is a preferred capsid for intramuscular (IM) injection. AAV1.1 (rAAV1/T265del) is an AAV1-derived capsid with enhanced skeletal muscle expression. AAV1.1 was employed to package a CNTFRα cDNA/CBA promotor vector and the vector was injected into hindlimb muscles of 120 d old SOD1^{G93A} mice, delaying end stage paralysis (FIG. 3) and slowing motor decline (FIG. 4).

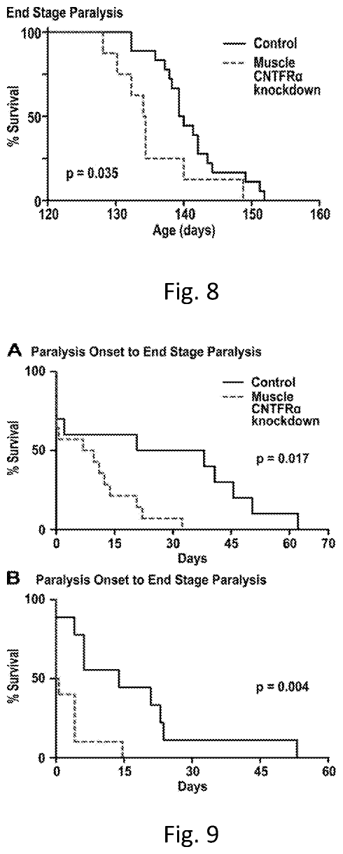

As shown in FIG. 3, AAV1.1-CNTFRα treatment delayed end stage paralysis. 2-tailed log rank p value shown (AAV1.1-CNTFRα, 173.0±3.7d, n=14; controls run in parallel, 161.1±2.0d, n=26, p=0.0033). The different control disease time course here vs. FIG. 8 reflects the mixed background for the knockdown study in FIG. 8 vs. the pure B6 background here. Control values here are consistent with previous pure B6 SOD1^{G93A} studies.

As shown in FIG. 4, AAV1.1-CNTFRα slowed decline in motor function accessed by hind limb grip strength (top panel) and rotarod performance (bottom panel), (tested every 2 wks starting at 11 wks except 17 wk to avoid acute effects of injection surgery; injection time indicated by arrows). ANOVA p values (p<0.0001) shown for main effect of treatment (control, n=15; AAV1.1-CNTFRα, n=14).

The observed effect is greater than many reported for this aggressive ALS model, but much more importantly, treatment began well after symptom onset (˜1.5 wks after max weight [widely used index of symptom onset; 109.2±1.6d here], and >4 wks after grip strength starts declining, FIG. 4, top panel). None of the many other reported SOD1^{G93A} treatments have been shown to work at all if started this late (whether compared in absolute terms, relative to symptom onset, or as % of control end stage age). Thus, the effect is much greater than any other at this clinically relevant time point, since no other treatment has been shown to be at all effective so late in the disease process. Given the clinical need for treatment well after symptom onset, this treatment is arguably the most promising to date.

Weight loss is the most sensitive measure of CNTF related side effects. AAV1.1-CNTFRα did not produce the weight loss seen with CNTF, since unlike CNTF injection, AAV1.1-CNTFRα enhances an endogenous anti-ALS response (ALS-induced muscle CNTFRα increase). Instead, AAV1.1-CNTFRα actually delayed disease-associated SOD1^{G93A} weight loss (20% wt loss age: AAV1.1-CNTFRα, 163.9±2.3d; control, 157.1±1.5d; p<0.03), and did not affect weight or even rate of weight gain in wild type mice (AAV1.1-CNTFRα mice=97±3% of control weight gain at 15 wks post-injection; n=4 pair).

The above AAV1.1-CNTFRα had a vector genome with a rat CNTFRα cDNA. This cDNA was replaced with a codon optimized mouse CNTFRα (mCNTFRα) cDNA (for host-vector species match and increased protein expression). The modified vector genome was packaged in AAV1.1 as a small-scale prep, and tested in the same above study. Like the rat version, it extended survival (grip strength and rotarod not tested) and may be even more effective (end stage: AAV1.1-mCNTFRα, 176.9±4.9d, n=7; parallel controls, 161.1±2.0d, n=26, p=0.007). Again, the treatment delayed ALS weight loss (AAV1.1-mCNTFRα 20% wt loss=171.3±4.1d, n=7 vs control 157.1±1.5d, n=26, p=0.0002), the opposite of CNTF side effects.

### Example 4: CNTFRα is Released from Muscle In Vivo and Contributes to Circulating CNTFRα Levels

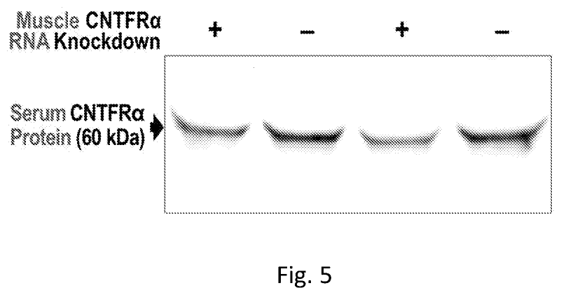

Muscle-specific CNTFRα depletion (with mlclf-Cre and floxed CNTFRα mice) and subsequent western blot analysis of circulating CNTFRα levels was used to show that CNTFRα is released from muscle in vivo and contributes to circulating CNTFRα levels. Results are shown in FIG. 5. Muscle-specific CNTFRα depletion also worsens ALS in a wide variety of ALS mice (see Example 7; FIGS. 8-10).

These data indicate that endogenous CNTFRα is released from muscle into the circulation in vivo and provide further evidence that CNTFRα released from muscle into the circulation inhibits ALS. The data further evidence that the disclosed AAV treatment works by increasing circulating CNTFRα. The data show that directly increasing circulating CNTFRα levels by IV infusion of CNTFRα protein (or with AAV vectors that increase circulating CNTFRα levels) inhibits ALS.

### Example 5: Enhancing Muscle CLC Well after Symptom Onset Slows SOD1^{G93A} Disease

Combined depletion of adult MN and muscle CNTFRα leads to MN terminal loss, but knockout of CNTF has no such effect, suggesting a ligand other than CNTF is involved in CNTFRα's effects, and suggesting this other ligand may also be involved in muscle CNTFRα's anti-ALS effects (consistent with negative CNTF ALS genetics). When cardiotrophin-like cytokine (CLC) and CNTFRα are heterologously expressed together in vitro, the cells release a soluble CLC/CNTFRα complex which can activate MN CNTF receptors and promote embryonic MN survival in vitro. Embryonic muscle cells express CLC and CNTFRα in vivo, suggesting they release CLC/CNTFRα to protect embryonic MNs from development related death. In agreement, knockout of either CLC or CNTFRα (but not CNTF) leads to embryonic MN loss. Moreover, adult muscle CLC is induced in all the ALS models similar to CNTFRα (increased 2.4±0.3 fold; n=8 in SOD1^{G93A} mice, undetectable in controls, and ˜2 fold above detection threshold in 1 yr TDP-43^{Q331K} mice; n=5 and increased 6.9±1.5 fold; n=3 in late stage SOD1^{G37R} mice).

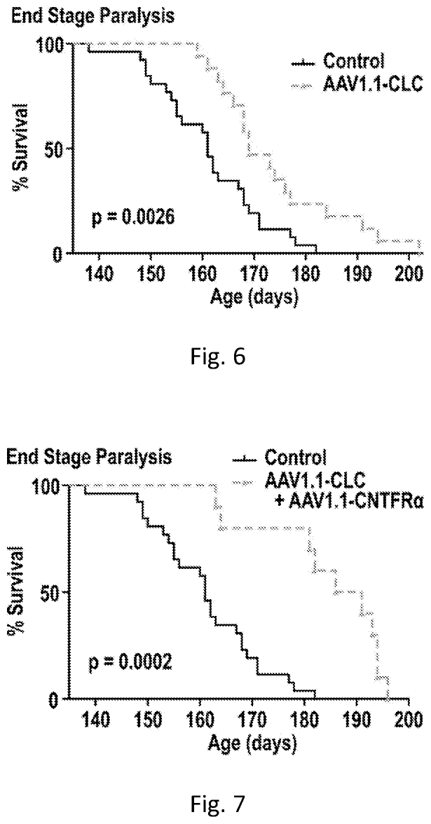

All the above indicate muscle CLC may be involved in CNTFRα's anti-ALS effects. Thus, increasing muscle CLC may be similarly therapeutic and again avoid side effects by enhancing an endogenous response (the muscle CLC RNA increase in ALS), and selectively targeting cells that express CLC (i.e., muscle) to potentially exploit endogenous mechanisms regulating release. To test the hypothesis, the vector's CNTFRα cDNA was replaced with a codon optimized mouse CLC cDNA and tested in the same study design as outlined above for AAV1.1-CNTFRα. AAV1.1-CLC (3×10¹⁰vg) or vehicle was injected bilaterally into gastrocnemius and soleus muscles of randomly assigned SOD1^{G93A} littermates at 120 days of age. AAV1.1-CLC treatment similarly delayed end stage paralysis (FIG. 6) despite being injected well after symptom onset, indicating AAV1.1-CLC is a very promising new ALS therapeutic. Like AAV1.1-CNTFRα, AAV1.1-CLC did not produce any sign of side effects, delayed ALS weight loss (20% wt loss: AAV1.1-CLC, 162.5±1.8d; control, 157.1±1.5; p<0.03), and did not affect weight gain in wild type mice (AAV1.1-CLC mice=107.6±8.1% of control wt gain at 15 wks post-injection; n=4).

Like the CNTFRα vector, the CLC vector did not produce any vector-derived RNA in spinal cord or rostral (triceps brachii) muscle. Results showed no AAV1.1-CLC-derived CLC RNA in heart and only trace amounts in liver (at least 6,000 fold less than in GAS). Similarly, results showed no AAV1.1-CNTFRα-derived CNTFRα RNA in heart and only trace levels in liver (at least 2000 fold less than in GAS). This similar muscle selectivity with CLC and CNTFRα vectors was expected since the capsid, promotor, and injection procedure, which all determine expression, were the same in all cases.

### Example 6: Combined AAV1.1-CNTFRα and AAV1.1-CLC Treatment Inhibits ALS in SOD1^{G93A} Mice

Several lines of data suggest CLC and CNTFRα are released as a MN protective CLC/CNTFRα complex. If AAV1.1-CNTFRα is therapeutic by enhancing CLC/CNTFRα production, this should be ultimately limited by the amount of available endogenous CLC. Similarly, a AAV1.1-CLC therapeutic effect should be limited by the amount of endogenous CNTFRα. If so, an AAV-induced increase in both CLC and CNTFRα may be most effective.

To test this hypothesis, a 1:1 ratio of AAV1.1-CNTFRα and AAV1.1-CLC was tested in the same study protocols outlined above. Combined treatment or vehicle was injected bilaterally into gastrocnemius and soleus muscles of randomly assigned female SOD1^{G93A} littermates at 120 days of age. While an improved effect was not observed in males over the individual treatments, the data showed this simple ratio enhances effect in females (FIG. 7). Median survival was 188.5 days, almost a month longer than controls (161 days), a remarkably large effect in this rapidly degenerating model, even compared to other treatments started much earlier in disease. These results were particularly exceptional, since treatment was initiated well after symptom onset at a clinically relevant time point at which no other treatment is reported to have any effect. The observed effect was not attributed to increased dose, since the result was observed at all doses tested in this pilot, including lower total doses than those used in the individual vector studies (mean survival: 3×10¹⁰vg (standard dose) of each vector=183.0d, n=3; 1×10¹⁰vg of each vector=186.5d, n=4; 3×10⁹vg of each vector=183d, n=3). The treatment did not lead to any sign of side effect and again delayed ALS weight loss (20% wt loss age: AAV1.1-CNTFRα/AAV1.1-CLC, 172.1±2.4d; control, 157.1±1.5; p<0.01).

The above data show that the AAV1.1-CLC vector increases CLC RNA locally in injected muscles but has a global therapeutic effect (i.e., protects muscles and motor neuron function in parts of the nervous system [rostral muscles and rostral motor neurons] that are not directly affected by the increased CLC RNA). This effect likely correlates with that observed for CNTFRα (release from muscle into sera to act globally) since CLC and CNTFRα are related proteins known to associate with each other and act together on motor neurons and be released together from muscle.

The data further suggest the utility of IV administration of a fusion protein comprising CLC covalently linked to CNTFRα via a flexible polypeptide linker sequence.

Similarly, the data suggest that ALS can be treated with IM administration of an AAV vector comprising a cDNA that encodes the appropriate precursor protein leading to the cellular secretion of a fusion protein made up of CLC covalently linked via a flexible polypeptide linker sequence to CNTFRα.

Further, the CLC data in combination with the CNTFRα data evidence that IV administration of CLC protein, either alone or in combination with CNTFRα protein, may have utility for treating ALS.

The data also suggest that CLF, another protein that acts on CNTF receptors and associates with CNTFRα and CLC, would be expected to be similarly effective when administered IV. Further, CLF would be expected to be effective when administered with CLC, since CLC and CLF associate together to activate CNTF receptors.

### Example 7: Endogenous Muscle CNTFRα Broadly Inhibits the Effects of Denervation and ALS-Inducing Genes

CNTF receptors contain CNTFRα, leukemia inhibitory factor receptor 0 and gp130. Only CNTFRα is unique to CNTF receptors, required for all CNTF receptor signaling, and not involved in other signaling. Thus, CNTFRα disruption best identifies in vivo CNTF receptor functions.

Neuromuscular CNTFRα is restricted to MNs and muscle. Muscle-specific CNTFRα depletion (with mlclf-Cre and foxed CNTFRα mice) does not affect MN axons or motor function in naïve mice but impairs MN axon regeneration and motor function recovery after nerve crush. Thus, muscle CNTFRα takes on a neuroregenerative/neuroprotective role after the denervating insult. This is not an indirect effect on muscle health since muscle atrophy, regeneration, fiber type and contractility were unaffected by the CNTFRα depletion. Instead, the data suggest muscle CNTFRα promotes MN axon recovery from denervating insults, and the increased muscle CNTFRα expression induced by nerve lesion and seen in denervating human diseases, including ALS, is a MN protective response to the denervating insults.

To test muscle CNTFRα's role in ALS, CNTFRα was specifically depleted in the most widely used ALS model, “high copy number” SOD1^{G93A} mice (Jax. #004435). CNTFRα-depleted (“knockdown”) mice and littermate controls (sex matched as possible) were run in parallel by investigators kept unaware of group. Both sexes were used. Quantitative real time RT-PCR (qRT-PCR) was normalized with GAPDH. End stage paralysis was defined as inability to right in 30 sec when placed on side. SOD1^{G93A} mice were tested biweekly from 10 wks of age, and then daily after initial paralysis. Muscle-specific CNTFRα knock-down led to earlier end stage paralysis and accelerated SOD1G93A disease (FIG. 8). Thus, results indicate endogenous muscle CNTFRα reduces the effect of this human ALS-inducing mutation. Moreover, the data likely underestimate muscle CNTFRα's impact, since this knockdown leaves ˜20% of muscle CNTFRα expression intact.

Mlclf-Cre and HSA-MCM-Cre were then used to specifically reduce muscle CNTFRα in SOD1^{G37R} mice (Jax #008229) which have a different human ALS mutation, less mutant SOD1 and late adult disease onset, as in human ALS.

As shown in FIG. 9, muscle-specific CNTFRα knockdown with mlclf-Cre (FIG. 9A) (n=10 control, 14 knockdown); and adult onset (2 month) muscle-specific CNTFRα knock-down with HSA-MCM-Cre (FIG. 9B) (n=9 control, 10 knock-down) both greatly accelerated SOD1^{G37R} paralysis onset to end stage (2-tailed log rank p values shown). Mice scored as “0 days” progressed from paralysis onset to end stage within the ½ wk interval between measurements. Mlclf-Cre and HSA-MCM-Cre experiments were run on different genetic backgrounds (since mlclf-Cre and HSA-MCM-Cre genes were obtained on different backgrounds) likely accounting for different control results.

Both CNTFRα depletion methods greatly accelerated disease in the previously defined paralytic final phase (FIG. 9). This phase is long after symptom onset (>100d after max wt and start of rotarod/grip strength decline in this slower model), analogous to when ALS patients are finally diagnosed and treatable. Thus, the data suggest muscle CNTFRα can inhibit ALS in patients that are diagnosed and treatable. The smaller knockdown effect in SOD1^{G93A} disease vs. the larger effect specific to later stage SOD1^{G37R} disease, mirrors CNTFRα's level of induction: 2.2±0.2 fold (n=8) muscle CNTFRα RNA increase in SOD1^{G93A} disease and a 7.8±0.6 fold (n=3) increase restricted to the late stage SOD1G37R disease (i.e., as expected, CNTFRα is most effective when most induced).

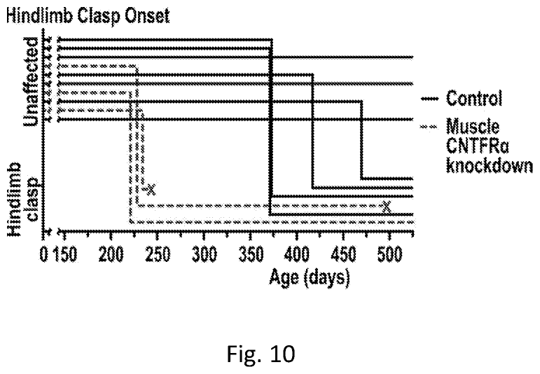

Transactivating response region DNA binding protein-43 (TDP-43) mutations cause 1-5% of familial ALS. TDP-43+ aggregates in all sporadic ALS suggest broad ALS involvement. Muscle CNTFRα knockdown greatly accelerated the hindlimb clasp motor deficit in TDP-43^{Q331K} mice (FIG. 10; p=0.0008), the only available mouse model with a human ALS-causing TDP-43 mutation and an adult-onset (like ALS), progressive motor deficit (Jax #017933). Moreover, 2 of the 3 knockdown mice reached end stage paralysis not seen at any age in TDP-43^{Q331L} mice without CNTFRα knockdown, indicating muscle CNTFRα not only greatly slows the disease but qualitatively reduces the maximum deficit. The TDP-43^{Q331K} CNTFRα knockdown mice remaining at 1 year also had larger rotarod and grip strength deficits (73.8±14.5% and 79.6±13.8% worse than the TDP-43^{Q331K} controls respectively). The CNTFRα knockdown does not affect any of the above indexes in naïve mice, so it accelerated TDP-43^{Q331K} disease, indicating muscle CNTFRα protects against this human ALS-inducing mutation. Muscle CNTFRα RNA expression is induced 4.4±0.6 fold (n=5) in this model by 1 year.

### Example 8: BDNF and NT-3 Treatments LED to Increased Survival in CLC-Treated SOD1^{G93A} ALS Mice

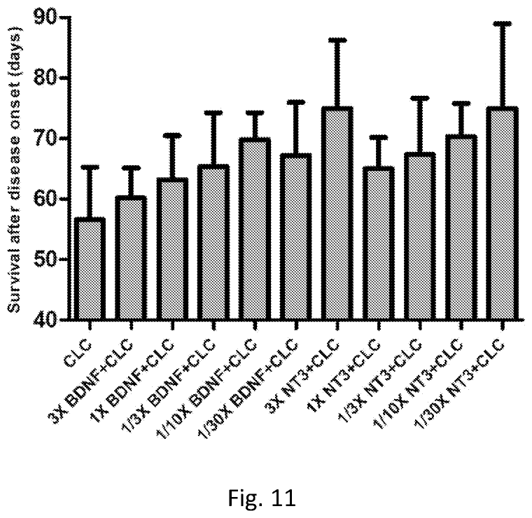

AAV1.1-CLC (3×10¹⁰vg/hindlimb) alone or in combination with different doses of either AAV1.1-BDNF or AAV1.1-NT3 was injected bilaterally into gastrocnemius and soleus muscles of randomly assigned SOD1^{G93A} littermates at 120 days of age, well after disease onset. As shown in FIG. 11, relative to the CLC treatment alone, both BDNF and NT-3 treatments increased mean survival following disease onset (onset=age at maximum weight; death=end stage paralysis defined as inability to right in 30 seconds when placed on either side). This increased therapeutic effect was observed across a wide range of AAV1.1-BDNF and AAV1.1-NT3 doses (shown relative to the CLC dose [i.e., 1×=3×10¹⁰vg]). Gehan-Breslow-Wilcoxon survival analysis test: Chi square=4.629; df=1; p=0.0314 comparing combination treatment mice to the CLC treatment alone mice. All mice run in parallel. Means+/−SEM presented. N=5 for CLC alone group and all CLC+BDNF groups; N=3 for all CLC+NT3 groups except 1/30×NT3+CLC where N=2.

### Example 9: BDNF and NT-3 Treatments LED to Increased Survival in CNTFRα-Treated SOD1^{G93A} ALS Mice

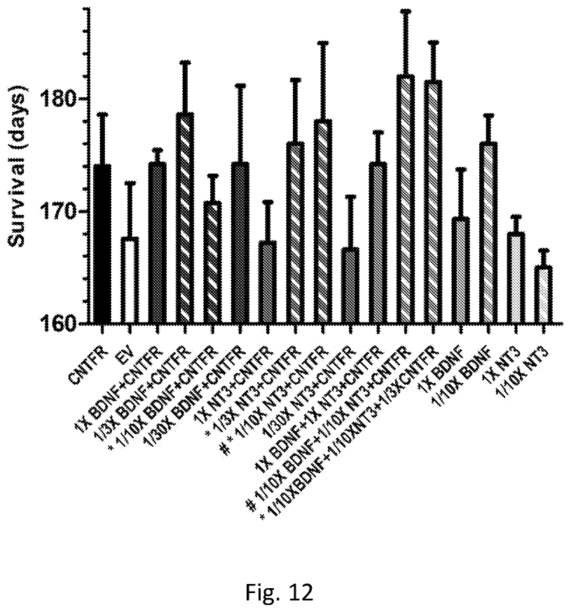

AAV1.1-CNTFRα (3×10¹⁰vg/hindlimb) alone or this CNTFRα treatment plus different doses of AAV1.1-BDNF and/or AAV1.1-NT3 was injected bilaterally into gastrocnemius and soleus muscles of randomly assigned SOD1^{G93A} littermates at 120 days of age, well after disease onset. As shown in FIG. 12, mice who received the regular CNTFRα treatment [i.e., 1×=3×10¹⁰vg/hindlimb] combined with BDNF or NT-3 treatments in the ⅓- 1/10× dose range survived longer (death=end stage paralysis defined as inability to right in 30 seconds when placed on either side) than those treated with CNTFRα alone. Those treated with a combination of CNTFRα and both BDNF and NT-3 in the ⅓- 1/10× dose range also survived longer on average than those treated with CNTFRα alone, even with ⅓ the regular CNTFRα dose. This average added survival time over control mice was approximately twice as large as the average added survival time over control for mice treated with CNTFRα alone. Mice treated with BDNF alone in this ⅓- 1/10× dose range also survived longer on average than the control mice, whereas this was not true for NT-3 treated mice at either dose tested. EV=empty AAV vector injected (3×10¹⁰vg/hindlimb) controls. CNTFR=CNTFRα. All mice run in parallel. Means+/−SEM presented. N=7 for CNTFRα alone and EV groups; N=5 for all CNTFRα+BDNF and CNTFRα+NT-3 groups except N=4 for 1/10×BDNF+ CNTFRα and 1/10×NT-3+ CNTFRα; N=5 for 1×BDNF+1×NT-3+ CNTFRα; N=4 for 1/10×BDNF+ 1/10×NT-3+ CNTFRα; N=2 for 1/10×BDNF+ 1/10×NT-3+⅓×CNTFRα; N=3 for all BDNF or NT-3 alone groups. *=excludes 1 mouse that did not display the typical, expected ALS-like paralysis of this ALS model. #=includes 1 mouse still alive as of the filing date of the instant disclosure. The average survival is at least as long as that shown here, and potentially substantially greater.

1. A method of treating a subject suffering from a motor neuron degenerative disorder, the method comprising administering to the subject one or more modified adeno-associated virus (AAV) vectors comprising a recombinant AAV (rAAV)-based genome, wherein the rAAV-based genome comprises one or more of: a brain derived neurotrophic factor (BDNF)-encoding cDNA insert or a neurotrophin-3 (NT-3)-encoding cDNA insert.

2. The method according to claim 1, wherein the BDNF-encoding cDNA insert encodes a protein having at least 80%, at least 85%, at least 90%, at least 95%, at least 96%, at least 97%, at least 98%, at least 99%, or 100% identity with SEQ ID NO: 14 or SEQ ID NO: 16.

3. The method according to claim 1, wherein the NT-3-encoding cDNA insert encodes a protein having at least 80%, at least 85%, at least 90%, at least 95%, at least 96%, at least 97%, at least 98%, at least 99%, or 100% identity with SEQ ID NO: 18 or SEQ ID NO: 20.

4. The method according to claim 1, further comprising administering to the subject one or more additional modified AAV vectors comprising an rAAV-based genome, wherein the rAAV-based genome comprises one or more of: a ciliary neurotrophic factor receptor alpha (CNTFRα)-encoding cDNA insert, a cardiotrophin-like cytokine (CLC)-encoding cDNA insert, a cytokine receptor-like factor 1 (CLF)-encoding cDNA insert, or a CNTFRα-CLC fusion protein-encoding cDNA insert.

5. The method according to claim 4, wherein the CNTFRα-encoding cDNA insert encodes a protein having at least 80%, at least 85%, at least 90%, at least 95%, at least 96%, at least 97%, at least 98%, at least 99%, or 100% identity with SEQ ID NO: 2 or SEQ ID NO: 4.

6. The method according to claim 4, wherein the CLC-encoding cDNA insert encodes a protein having at least 80%, at least 85%, at least 90%, at least 95%, at least 96%, at least 97%, at least 98%, at least 99%, or 100% identity with SEQ ID NO: 6 or SEQ ID NO: 8.

7. The method according to claim 4, wherein the CLF-encoding cDNA insert encodes a protein having at least 80%, at least 85%, at least 90%, at least 95%, at least 96%, at least 97%, at least 98%, at least 99%, or 100% identity with SEQ ID NO: 10 or SEQ ID NO: 12.

8. The method according to claim 4, wherein the CNTFRα-CLC fusion protein-encoding cDNA insert encodes a protein having at least 80%, at least 85%, at least 90%, at least 95%, at least 96%, at least 97%, at least 98%, at least 99%, or 100% identity with SEQ ID NO: 21 or SEQ ID NO: 22.

9. The method according to claim 1, wherein the motor neuron degenerative disorder is amyotrophic lateral sclerosis (ALS).

10. The method according to claim 9, wherein the ALS is late-stage ALS.

11. The method according to claim 10, wherein the subject is considered to be suffering from late-stage ALS when the subject requires a gastrostomy, requires non-invasive ventilation, and/or experiences clinical symptoms in at least three CNS regions of the body.

12. The method according to claim 9, wherein the ALS is characterized by one or both of: at least one TDP-43 mutation; and an abnormal cellular TDP-43 distribution.

13. The method according to claim 1, wherein the rAAV-based genome is modified to be muscle-tropic.

14. The method according to claim 1, wherein administering comprises non-systemic administration.

15. The method according to claim 14, wherein non-systemic administration comprises intramuscular (IM) administration to one or more muscles selected from non-respiratory skeletal muscles, respiratory muscles, and combinations thereof.

16. The method according to claim 1, further comprising administering to the subject an effective amount of a docosahexanoic acid (DHA) dietary supplement.

17. A pharmaceutical composition comprising:
one or more muscle-tropic modified AAV vectors, each of said AAV vectors comprising an rAAV genome, each of said rAAV genomes engineered to comprise:
(i) one or more of: a BDNF-encoding cDNA insert or an NT-3-encoding cDNA insert; and
(ii) a promoter; and
at least one pharmaceutically-acceptable excipient.

18. The pharmaceutical composition according to claim 17, further comprising one or more additional muscle-tropic AAV vectors comprising an rAAV genome engineered to comprise one or more of: a CNTFRα-encoding cDNA insert, a CLC-encoding cDNA insert, a CLF-encoding cDNA insert, or a CNTFRα-CLC fusion protein-encoding cDNA insert.

19. The pharmaceutical composition according to claim 17, wherein the composition is formulated for intramuscular administration or intravenous administration.

20. A modified AAV vector comprising a plurality of recombinant AAV (rAAV)-based genomes, wherein the rAAV genomes comprise:
at least one of: a BDNF-encoding cDNA insert or an NT-3-encoding cDNA insert; and
one or more of:
a CNTFRα-encoding cDNA insert;
a CLC-encoding cDNA insert;
a CLF-encoding cDNA insert; and
a CNTFRα-CLC fusion protein-encoding cDNA insert.
